(19) United States
(12) Patent Application Publication
(10) Pub. No.: US 2021/0275439 A1
(43) Pub. Date: Sep. 9, 2021

(54) FLUTICASONE AND VILANTEROL FORMULATION AND INHALER

(71) Applicant: KINDEVA DRUG DELIVERY L.P., St. Paul, MN (US)

(72) Inventors: Sarah J. Dexter, St. Paul, MN (US); Philip M. Cocks, St. Paul, MN (US)

(21) Appl. No.: 17/059,617

(22) PCT Filed: Jun. 5, 2019
(86) PCT No.: PCT/US2019/035489

§ 371 Date: Nov. 30, 2020

Related U.S. Application Data
(60) Provisional application No. 62/681,980, filed on Jun. 7, 2018.

(51) Int. Cl. A61K 9/00; A61K 31/56; A61K 31/138; A61K 31/02

(52) U.S. Cl. CPC A61K 9/008; A61K 31/02; A61K 31/138; A61K 31/56

(57) ABSTRACT

A composition comprising particulate fluticasone or a pharmaceutically acceptable salt or solvate thereof, particulate vilanterol or a pharmaceutically acceptable salt or solvate thereof, and 1,1-difluoroethane.

### FIELD

The present disclosure generally relates to formulations used for, as an example, inhaled dosage forms, as well as aerosol canisters, inhalers, such as metered dose inhalers, containing the same. In particular, the present disclosure relates to formulations including fluticasone and vilanterol.

### BACKGROUND

Currently, dry-powder inhalers (DPIs) containing fluticasone furoate and vilanterol trifenatate are commercially available. These include GlaxoSmithKline's Relvar® Ellipta® and Breo® Ellipta® DPIs.

### SUMMARY

Pressurized metered dose inhalers (pMDIs) can have several advantages over DPIs. For example, controlling the stability of the micronized medicament in a DPI so that it delivers a consistent dose to a patient can be challenging. Also, in some instances, pMDI manufacture may be done less expensively than a DPI product.

According to the present disclosure, there is provided a composition comprising particulate fluticasone or a pharmaceutically acceptable salt or solvate thereof, particulate vilanterol or a pharmaceutically acceptable salt or solvate thereof, and 1,1-difluoroethane (HFA-152a).

In embodiments, the fluticasone or a pharmaceutically acceptable salt or solvate thereof can be fluticasone furoate.

In embodiments, the vilanterol or a pharmaceutically acceptable salt or solvate thereof is vilanterol trifenatate.

In embodiments, the propellant can comprise or consist essentially of 1,1-difluoroethane (HFA-152a).

In embodiments, the canister size of the fluticasone can be between about 2 micrometers and 4 micrometers.

In embodiments, the canister size of the vilanterol trifenatate can be between about 1 micrometer and 2 micrometers.

In embodiments, the concentration of the fluticasone can be between about 1.0 mg/g and 2.5 mg/g.

In embodiments, the concentration of the fluticasone can be between about 2.0 mg/g and 4.5 mg/g.

In embodiments, the concentration of the vilanterol trifenatate can be between about 0.2 mg/g and 1.0 mg/g.

Further, according to the present disclosure, there is provided a composition comprising particulate fluticasone or a pharmaceutically acceptable salt or solvate thereof, particulate vilanterol or a pharmaceutically acceptable salt or solvate thereof, and 1,1-difluoroethane (HFA-152a), wherein fluticasone and vilanterol or the pharmaceutically acceptable salts or solvates thereof are the only active medicaments in the composition.

In embodiments, the fluticasone or a pharmaceutically acceptable salt or solvate thereof can be fluticasone furoate.

In embodiments, the vilanterol or a pharmaceutically acceptable salt or solvate thereof is vilanterol trifenatate.

In embodiments, the propellant can comprise or consist essentially of 1,1-difluoroethane (HFA-152a).

In embodiments, the canister size of the fluticasone can be between about 2 micrometers and 4 micrometers.

In embodiments, the canister size of the vilanterol trifenatate can be between about 1 micrometer and 2 micrometers.

In embodiments, the concentration of the fluticasone can be between about 1.0 mg/g and 2.5 mg/g.

In embodiments, the concentration of the fluticasone can be between about 2.0 mg/g and 4.5 mg/g.

In embodiments, the concentration of the vilanterol trifenatate is between about 0.2 mg/g and 1.0 mg/g.

Further, according to the present disclosure, there is provided an aerosol canister comprising a composition of the disclosed embodiments.

In embodiments, the aerosol canister can include at least one surface having a primer composition comprising a silane having two or more reactive silane groups separated by an organic linker group disposed thereon, wherein the primer composition has a coating composition comprising an at least partially fluorinated compound disposed thereon.

In embodiments, the at least partially fluorinated compound is a polyfluoropolyether silane.

In embodiments, the at least one surface is at least a portion of a valve surface.

Further, according to the present disclosure, there is provided an inhaler of comprising the composition of any one of the disclosed embodiments or the aerosol canister of any one of the disclosed embodiments.

Other features and aspects of the present disclosure will become apparent by consideration of the detailed description.

### DETAILED DESCRIPTION

Throughout this disclosure, singular forms such as “a,” “an,” and “the” are often used for convenience; singular forms are meant to include the plural unless the singular alone is explicitly specified or is clearly indicated by the context. Numerical ranges, for example “between x and y” or “from x to y”, include the endpoint values of x and y.

Some terms used in this application have special meanings, as defined herein. All other terms will be known to the skilled artisan, and are to be afforded the meaning that a person of skill in the art at the time of the invention would have given them.

Elements in this specification that are referred to as “common” and “commonly used” and similar descriptors should be understood to be common within the context of the compositions, articles, such as inhalers and metered dose inhalers, and methods of this disclosure; this terminology is not used to mean that these features are present, much less common, in the prior art. Unless otherwise specified, only the Background section of this Application refers to the prior art.

The “particle size” of a single particle is the size of the smallest hypothetical hollow sphere that could encapsulate the particle.

The “mass median diameter” or MMD of a plurality of particles refers to the value for a particle diameter at which 50% of the mass of particles in the plurality of particles have a particle size smaller than the value and 50% of the mass of particles in the plurality of particle have a particle size greater than the value.

The “canister size” of a plurality of particles refers to the mass mean diameter of the plurality of particles when the formulation is prepared.

The “ex-actuator size” of a plurality of particles refers to the mass median aerodynamic diameter (or MMAD) of the plurality of particles after the plurality of particles has passed through the actuator of an inhaler, such as a metered dose inhaler, as measured by the procedure described in the United States Pharmacopeia <601>.

When the concentration of fluticasone is discussed in this application, for convenience it is referred to in terms of the concentration of the form of fluticasone that is most commonly used in this disclosure, that is, fluticasone furoate. It should therefore be understood that if another form or salt of fluticasone is used, the concentration of that other form or salt should be calculated on a basis relative to fluticasone furoate. A person of ordinary skill in the relevant arts can easily perform this calculation by comparing the molecular weight of the form or salt of fluticasone that is used to the molecular weight of fluticasone furoate.

When the formulation concentration of vilanterol is discussed in this application, for convenience it is referred to in terms of the concentration of the form of vilanterol that is most commonly used in this disclosure, that is, vilanterol trifenatate, unless otherwise specified. It should therefore be understood that if another form or salt of vilanterol is used, the formulation concentration of that other form or salt should be calculated on a basis relative to vilanterol trifenatate. A person of ordinary skill in the relevant arts can easily perform this calculation by comparing the molecular weight of the form or salt of vilanterol that is used to the molecular weight of vilanterol trifenatate. When doses of vilanterol are discussed, such as nominal product doses or measured delivered doses, they are given for vilanterol base unless otherwise specified.

### Formulation

A pharmaceutical formulation comprises particulate fluticasone. The fluticasone can be a free base, but can be in the form of one or more physiologically acceptable salts or solvates, such as fluticasone furoate and fluticasone propionate.

The fluticasone, such as fluticasone furoate, can be in particulate form. The canister size of the particles of fluticasone, such as fluticasone furoate, can be any suitable canister size. Exemplary suitable canister sizes can be no less than 1 micrometer, no less than 1.5 micrometers, no less than 2 micrometers, no less than 2.5 micrometers, no less than 3 micrometers, no less than 3.5 micrometers, no less than 4 micrometers, or no less than 4.5 micrometers. Exemplary suitable canister sizes can also be no greater than 5 micrometers, no greater than 4.5 micrometers, no greater than 4.0 micrometers, no greater than 3.5 micrometers, no greater than 3.0 micrometers, no greater than 2.5 micrometers, no greater than 2.0 micrometers, or no greater than 1.5 micrometers. 1 micrometer to 5 micrometers is common In embodiments the canister size may be between 2.0 and 4.0 micrometers. In embodiments the canister size may be between 2.0 and 3.0 micrometers.

The ex-actuator size of the fluticasone particles, such as fluticasone furoate particles, can be any suitable ex-actuator size. Exemplary suitable ex-actuator sizes can be no less than 1 micrometer no less than 1.5 micrometers, no less than 2 micrometers, no less than 2.5 micrometers, no less than 3 micrometers, no less than 3.5 micrometers, no less than 4 micrometers, or no less than 4.5 micrometers. Exemplary suitable ex-actuator sizes can also be no greater than 5 micrometers, no greater than 4.5 micrometers, no greater than 4.0 micrometers, no greater than 3.5 micrometers, no greater than 3.0 micrometers, no greater than 2.5 micrometers, no greater than 2.0 micrometers, or no greater than 1.5 micrometers. 1 micrometer to 5 micrometers is common In embodiments the ex-actuator size may be between 2.0 and 4.0 micrometers. In embodiments the ex-actuator size may be between 2.5 and 3.5 micrometers.

The fluticasone, such as fluticasone furoate, can be present in any suitable concentration in the formulation. When the concentration of fluticasone is expressed in terms of mg/g of formulation, then the concentration of fluticasone can be no less than 0.1, no less than 0.2, no less than 0.3, no less than 0.4, no less than 0.5, no less than 0.6, no less than 0.7, no less than 0.8, no less than 0.9, no less than 1.0, no less than 1.5, or no less than 2.0. Also on a mg/g basis, the concentration of fluticasone can be no greater than 10.0, no greater than 8.0, no greater than 6.0, no greater than 5.0, no greater than 4.0, no greater than 3.0, no greater than 2.5, no greater than 2.2, or no greater than 2.0. One exemplary range is from 1.0 mg/g to 2.5 mg/g. Another exemplary range is from 2.0 mg/g to 4.5 mg/g. Another exemplary range is from 1.0 mg/g to 5 mg/g. Another exemplary range is from 2.0 to 10.0 mg/g. For some applications, a concentration of about 1.7 mg/g is employed. For other applications, a concentration of about 3.5 mg/g is employed.

The composition also comprises vilanterol, such as vilanterol trifenatate. The vilanterol, such as vilanterol trifenatate, can also be in particulate form. The canister size of the particles of vilanterol, such as vilanterol trifenatate, can be any suitable canister size. Exemplary suitable canister sizes can be no less than 1 micrometer no less than 1.5 micrometers, no less than 2 micrometers, no less than 2.5 micrometers, no less than 3 micrometers, no less than 3.5 micrometers, no less than 4 micrometers, or no less than 4.5 micrometers. Exemplary suitable canister sizes can also be no greater than 5 micrometers, no greater than 4.5 micrometers, no greater than 4.0 micrometers, no greater than 3.5 micrometers, no greater than 3.0 micrometers, no greater than 2.5 micrometers, no greater than 2.0 micrometers, or no greater than 1.5 micrometers. 1 micrometer to 5 micrometers is common In embodiments the canister size may be between 3.0 and 4.5 micrometers. In embodiments the canister size may be between 1.0 and 2.0 micrometers.

The ex-actuator size of the vilanterol particles, such as vilanterol trifenatate, can be any suitable ex-actuator size. Exemplary suitable ex-actuator sizes can be no less than 1 micrometer no less than 1.5 micrometers, no less than 2 micrometers, no less than 2.5 micrometers, no less than 3 micrometers, no less than 3.5 micrometers, no less than 4 micrometers, or no less than 4.5 micrometers. Exemplary suitable ex-actuator sizes can also be no greater than 5 micrometers, no greater than 4.5 micrometers, no greater than 4.0 micrometers, no greater than 3.5 micrometers, no greater than 3.0 micrometers, no greater than 2.5 micrometers, no greater than 2.0 micrometers, or no greater than 1.5 micrometers. 1 micrometer to 5 micrometers is common In embodiments the ex-actuator size may be between 1.0 and 4.0 micrometers. In embodiments the ex-actuator size may be between 1.5 and 2.5 micrometers.

The vilanterol can be used in any suitable concentration. On a mg/g basis, exemplary concentrations are no less than 0.05, no less than 0.10, no less than 0.15, no less than 0.20, no less than 0.25, no less than 0.30, no less than 0.35, no less than 0.40, no less than 0.45, or no less than 0.5. Exemplary concentrations are also no greater than 2.0, no greater than 1.9, no greater than 1.8, no greater than 1.7, no greater than 1.6, no greater than 1.5, no greater than 1.4, no greater than 1.3, no greater than 1.2, no greater than 1.1, no greater than 1.0, no greater than 0.9, no greater than 0.8, no greater than 0.7, no greater than 0.6, or no greater than 0.5. Common concentrations are from 0.2 mg/g to 2.0 mg/g, such as 0.2 mg/g to 1.0 mg/g or 0.4 mg/g to 0.8 mg/g. For some applications, a concentration of 0.4 mg/g is used. For other applications, a concentration of 0.7 mg/g is used. For still other applications, a concentration of 0.9 mg/g is used.

In some embodiments, the fluticasone and vilanterol as described above can be the only active medicaments in the composition.

A propellant is also included in the formulation. The propellant can be 1,1-difluoroethane (also known as HFA-152a). In certain embodiments, the propellant can further comprise 1,1,1,2,3,3,3-heptafluoropropane (also known as HFA-227 or HFC-227), and/or 1,1,1,2-tetrafluoroethane (also known as HFA-134 or HFC-134), in combination with 1,1-difluoroethane (HFA-152a). In some embodiments the propellant consists essentially of 1,1-difluoroethane (HFA-152a). The propellant can also serve as a dispersant for the particles of fluticasone, such as fluticasone furoate, and vilanterol, such as vilanterol trifenatate.

The particles of fluticasone, such as fluticasone furoate, and vilanterol, such as vilanterol trifenatate, may not be dissolved in the formulation. Instead, the particles of fluticasone, such as fluticasone furoate, and vilanterol, such as vilanterol trifenatate are suspended in the propellant.

In some embodiments the composition consists essentially of fluticasone, vilanterol, and one or more propellants.

In order to facilitate this suspension, additional components can be added to the formulation. One such additional component is ethanol. Another such additional component is a surfactant. These additional components are not required unless otherwise specified.

When ethanol is used, it can be employed in relatively low concentrations. On a weight percent basis, the amount of ethanol used, if any, can be no greater than 5, no greater than 4.5, no greater than 4.0, no greater than 3.5, no greater than 3.0, no greater than 2.5, no greater than 2.0, no greater than 1.5, no greater than 1.4, no greater than 1.3, no greater than 1.2, no greater than 1.1, no greater than 1.0, no greater than 0.9, no greater than 0.8, no greater than 0.7, no greater than 0.6, or no greater than 0.5. On a weight percent basis, the amount of ethanol used, if any, can be no less than 0.5, no less than 0.6, no less than 0.7, no less than 0.8, no less than 0.9, no less than 1.0, no less than 1.1, no less than 1.1, no less than 1.2, no less than 1.3, no less than 1.4, no less than 1.5, no less than 2.0, no less than 2.5, no less than 3.0, no less than 3.5, no less than 4.0, no less than 4.5, or no less than 5.0. Exemplary ranges of ethanol concentration, in those cases when ethanol is included, are from 0.1 wt. % to 5 wt. %, such as from 0.5 wt. % to 4 wt. %. In some cases, an ethanol concentration of 1 wt. % is employed.

One or more surfactants can also be used to facilitate suspension of the particles in the formulation. However, surfactant-free formulations can be advantageous for some purposes, and surfactant is not required unless otherwise specified.

Any pharmaceutically acceptable surfactant can be used. Most such surfactants are suitable for use with an inhaler. Exemplary surfactants include oleic acid, sorbitan monooleate, sorbitan trioleate, soya lecithin, polyethylene glycol, polyvinylpyrrolidone, or combinations thereof. Oleic acid, polyvinylpyrrolidone, or a combination thereof is most common. A combination of polyvinylpyrrolidone and polyethylene glycol is also commonly employed. When polyvinylpyrrolidone is employed, it can have any suitable molecular weight. Examples of suitable weight average molecular weights are from 10 to 100 kilodaltons, and can be from 10 to 50, 10 to 40, 10 to 30 or 10 to 20 kilodaltons. When polyethylene glycol is employed, it can be any suitable grade. PEG 1000 and PEG 300 are most commonly employed.

When used, the surfactant can be present, on a weight percent basis, in an amount no less than 0.0001, no less than 0.01, no less than 0.02, no less than 0.03, no less than 0.04, no less than 0.05, no less than 0.06, no less than 0.07, no less than 0.08, no less than 0.09, no less than 0.10, no less than 0.15, no less than 0.20, no less than 0.25, no less than 0.3, no less than 0.4, no less than 0.5, no less than 0.6, no less than 0.7, no less than 0.8, no less than 0.9, or no less than 1. The surfactant can be present, on a weight percent basis, in an amount no greater than 1, no greater than 0.9, no greater than 0.8, no greater than 0.7, no greater than 0.6, no greater than 0.5, no greater than 0.4, no greater than 0.3, no greater than 0.25, no greater than 0.20, no greater than 0.15, no greater than 0.14, no greater than 0.13, no greater than 0.12, no greater than 0.11, no greater than 0.10, no greater than 0.09, no greater than 0.08, no greater than 0.07, no greater than 0.06, no greater than 0.05, no greater than 0.04, no greater than 0.03, no greater than 0.02, or no greater than 0.01. Concentration ranges can be from 0.0001 wt. % to 1 wt. %, such as 0.001 wt. % to 0.1 wt. %. Particular applications use 0.01 wt. % surfactant.

Particularly, oleic acid can be used in any of the abovementioned concentrations. Particularly, polyvinylpyrrolidone can be used in any of the abovementioned concentrations. Particularly, a combination of polyethylene glycol and polyvinylpyrrolidone can be used in any of the abovementioned concentrations. Particularly, sorbitan trioleate can be used in any of the abovementioned concentrations.

The above-described formulations can be used with metered dose inhalers known in the art.

Exemplary metered dose inhalers for the pharmaceutical formulations described herein contain an aerosol canister fitted with a valve. The canister can have any suitable volume. The brimful capacity canister will depend on the volume of the formulation that is used to fill the canister. In exemplary applications, the canister will have a volume from 5 mL to 500 mL, such as, for example 10 mL to 500 mL, 25 mL to 400 mL, 5 mL to 50 mL, 8 mL to 30 mL, 10 mL to 25 mL, or 5 to 20 mL. The canister will often have sufficient volume to contain enough medicament for delivering an appropriate number of doses. The appropriate number of doses is discussed herein. The valve can be affixed, or crimped, onto the canister by way of a cap or ferrule. The cap or ferrule is often made of aluminum or an aluminum alloy, which can be part of the valve assembly. One or more seals can be located between the canister and the ferrule. The seals can be one or more of O-ring seals or gasket seals. The valve can be a metered dose valve. Exemplary valve sizes range from 20 microliters to 100 microliters. Specific valve size that are commonly employed include 25, 50, 60, and 63 microliter valve sizes.

The container and valve can include an actuator. Most actuators have a patient port, which can be a mouthpiece, for delivering the formulation contained in the canister. The patient port can be configured in a variety of ways depending on the intended destination of the formulation. For example, a patient port designed for administration to the nasal cavities will generally have an upward slope to direct the formulation to the nose. The actuator is most commonly made of plastic material. Exemplary plastic materials for this purpose include at least one of polyethylene and polypropylene. Exemplary MDIs have an actuator with an orifice diameter. Any suitable orifice diameter can be used. Exemplary orifice diameters are from 0.2 mm to 0.65 mm. Exemplary orifice jet length is from 0.5 mm to 1.5 mm. Specific examples include orifice diameters of 0.2 mm, 0.25 mm, 0.3 mm, 0.4 mm, 0.5 mm, or 0.6 mm, any of which can have an orifice jet length of 0.8 mm, 1.0 mm, or 1.5 mm.

A metered dose valve can be present, and is often located at least partially within the canister and at least partially in communication with the actuator. Exemplary metered dose valves include a metering chamber that is at least partially defined by an inner valve body through which a valve stem passes. The valve stem can be biased outwardly by a compression spring to be in a sliding sealing engagement with an inner tank seal and outer diaphragm seal. The valve can also include a second valve body in the form of a body emptier. The inner valve body, which is sometimes referred to as the primary valve body, defines, in part, the metering chamber. The second valve body, which is sometimes referred to as the secondary valve body, defines, in part, a pre-metering region (sometimes called a pre-metering chamber) in addition to serving as a bottle emptier. The outer walls of the portion of the metered dose valve that are located within the canister, as well as the inner walls of the canister, defined a formulation chamber for containing the pharmaceutical formulation.

In use, the pharmaceutical formulation passes from the formulation chamber into the metering chamber. In moving to the metering chamber, the formulation can pass into the above-mentioned pre-metering chamber through an annular space between the secondary valve body (or a flange of the secondary valve body) and the primary valve body. Pressing the valve stem towards the interior of the container actuates the valve, which allows the pharmaceutical formulation to pass from the pre-metering chamber through a side hole in the valve stem, through an outlet in the valve stem, to an actuator nozzle, and finally through the patient port to the patient. When the valve stem is released, the pharmaceutical formulation enters the valve, such as to the pre-metering chamber, through an annular space and then travels to the metering chamber.

The pharmaceutical formulation can be placed into the canister by any known method. The two most common methods are cold filling and pressure filling. In a cold filling process, the pharmaceutical formulation is chilled to an appropriate temperature, which can be −40° C. to −60° C. for formulations that use propellant HFA 152a, HFA 134a, HFA 227, or a combination thereof, and added to the canister. The metered dose valve is subsequently crimped onto the canister. When the canister warms to ambient temperature, the vapor pressure associated with the pharmaceutical formulation increases thereby providing an appropriate pressure within the canister.

In a pressure filling method, the metered dose valve can be first crimped onto the empty canister. Subsequently, the formulation can be added through the valve into the container by way of applied pressure. Alternatively, all the non-volatile components can be first added to the empty canister before crimping the valve onto the canister. The propellant can then be added through the valve into the canister by way of applied pressure.

Upon actuation, exemplary metered dose inhalers that are filled with any one of the formulations described herein can produce a fine particle mass of vilanterol, particularly vilanterol that is from 5 mcg to 20 mcg per actuation and a fine particle mass of fluticasone, particularly fluticasone furoate, that is from 10 mcg to 40 mcg per actuation. In particular cases, inhalers, such as metered dose inhalers, produce a fine particle mass of vilanterol that is from 6 mcg to 12 mcg, and a fine particle mass of fluticasone, particularly fluticasone furoate, that is from 15 mcg to 25 mcg per actuation. In particular cases, inhalers, such as metered dose inhalers, produce a fine particle mass of vilanterol that is from 6 mcg to 12 mcg, and a fine particle mass of fluticasone, particularly fluticasone furoate, that is from 25 mcg to 35 mcg per actuation. Fine particle mass can be calculated by the procedure described in the experimental section of this disclosure.

The fine particle masses discussed above can correspond to a fine particle fraction of vilanterol and of fluticasone, particularly fluticasone furoate, that is from 20% to 65%, which can be from 20% to 40% in particular cases, or from 25% to 35% in more particular cases. Fine particle fraction can be calculated by the procedure described in the experimental section of this disclosure.

Exemplary metered dose inhalers are designed to deliver a specified number of doses of the pharmaceutical formulation. In most cases, the specified number of doses is from 15 to 400, such as from 120 to 250 or such as 15 to 60. One commonly employed metered dose inhaler is designed to provide 120 doses; this can be employed with any of the formulations or inhaler types described herein. Another commonly employed metered dose inhaler is designed to provide 240 doses; this can be employed with any of the formulations or inhaler types described herein. In another embodiment, a metered dose inhaler may provide 30 doses.

The metered dose inhaler can contain a dose counter for counting the number of doses. Suitable dose counters are known in the art, and are described in, for example, U.S. Pat. Nos. 8,740,014, 8,479,732, and 8,814,035, and U.S. Patent Application Publication No. 2012/0234317, all of which are incorporated by reference in their entirety with respect to their disclosures of dose counters.

One exemplary dose counter, which is described in detail in U.S. Pat. No. 8,740,014 (which is hereby incorporated by reference in its entirety for its disclosure of the dose counter) has a fixed ratchet element and a trigger element that is constructed and arranged to undergo reciprocal movement coordinated with the reciprocal movement between an actuation element in an inhaler and the dose counter. The reciprocal movement can comprise an outward stroke (outward being with respect to the inhaler) and a return stroke. The return stroke returns the trigger element to the position that it was in prior to the outward stroke. A counter element is also included in this type of dose counter. The counter element is constructed and arranged to undergo a predetermined counting movement each time a dose is dispensed. The counter element is biased towards the fixed ratchet and trigger elements and is capable of counting motion in a direction that is substantially orthogonal to the direction of the reciprocal movement of the trigger element.

The counter element in the above-described dose counter comprises a first region for interacting with the trigger member. The first region comprises at least one inclined surface that is engaged by the trigger member during the outward stroke of the trigger member. This engagement during the outward stroke causes the counter element to undergo a counting motion. The counter element also comprises a second region for interacting with the ratchet member. The second region comprises at least one inclined surface that is engaged by the ratchet element during the return stroke of the trigger element causing the counter element to undergo a further counting motion, thereby completing a counting movement. The counter element is normally in the form of a counter ring, and is advanced partially on the outward stroke of the trigger element, and partially on the return stroke of the trigger element. As the outward stroke of the trigger can correspond to the depression of a valve stem that causes firing of the valve (and, in the case of a metered dose inhaler, also meters the contents) and the return stroke can correspond to the return of the valve stem to its resting position, this dose counter allows for precise counting of doses.

Another suitable dose counter, which is described in detail in U.S. Pat. No. 8,479,732 (which is incorporated by reference in its entirety for its disclosure of dose counters) is specially adapted for use with a metered dose inhaler. This dose counter includes a first count indicator having a first indicia bearing surface. The first count indicator is rotatable about a first axis. The dose counter also includes a second count indicator having a second indicia bearing surface. The second count indicator is rotatable about a second axis. The first and second axes are disposed such that they form an obtuse angle. The obtuse angle mentioned above can be any obtuse angle, but is advantageously 125 to 145 degrees. The obtuse angle permits the first and second indicia bearing surface to align at a common viewing area to collectively present at least a portion of a medication dosage count. One or both of the first and second indicia bearing surfaces can be marked with digits, such that when viewed together through the viewing area the numbers provide a dose count. For example, one of the first and second indicia bearing surface may have “hundreds” and “tens” place digits, and the other with “ones” place digits, such that when read together the two indicia bearing surfaces provide a number between 000 and 999 that represents the dose count.

Yet another suitable dose counter is described in U.S. Patent Application Publication No. 2012/0234317 (hereby incorporated by reference in its entirety for its disclosure of dose counters). Such a dose counter includes a counter element that undergoes a predetermined counting motion each time a dose is dispensed. The counting motion can be vertical or essentially vertical. A count indicating element is also included. The count indicating element, which undergoes a predetermined count indicating motion each time a dose is dispensed, includes a first region that interacts with the counter element.

The counter element has regions for interacting with the count indicating element. Specifically, the counter element comprises a first region that interacts with a count indicating element. The first region includes at least one surface that it engaged with at least one surface of the first region of the aforementioned count indicating element. The first region of the counter element and the first surface of the count inducing element are disposed such that the count indicating member completes a count indicating motion in coordination with the counting motion of the counter element, during and induced by the movement of the counter element, the count inducing element undergoes a rotational or essentially rotational movement. In practice, the first region of the counter element or the counter indicating element can comprise, for example, one or more channels. A first region of the other element can comprise one or more protrusions adapted to engage with said one or more channels.

Yet another dose counter is described in U.S. Pat. No. 8,814,035 (hereby incorporated by reference in its entirety for its disclosure of dose counters). Such a dose counter is specially adapted for use with an inhaler with a reciprocal actuator operating along a first axis. The dose counter includes an indicator element that is rotatable about a second axis. The indicator element is adapted to undergo one or more predetermined count-indicating motions when one or more doses are dispensed. The second axis is at an obtuse angle with respect to the first axis. The dose counter also contains a worm rotatable about a worm axis. The worm is adapted to drive the indicator element. It may do this, for example, by containing a region that interacts with and enmeshes with a region of the indicator element. The worm axis and the second axis do not intersect and are not aligned in a perpendicular manner The worm axis is also, in most cases, not disposed in coaxial alignment with the first axis. However, the first and second axes may intersect.

At least one of the various internal components of an inhaler, such as a metered dose inhaler, as described herein, such as one or more of the canister, valve, gaskets, seals, or O-rings, can be coated with one or more coatings. Some of these coatings provide a low surface energy. Such coatings are not required because they are not necessary for the successful operation of all inhalers.

Some coatings that can be used are described in U.S. Pat. Nos. 8,414,956 and 8,815,325 and United States Patent Application Publication No. 2012/0097159, all of which are incorporated by reference in their entireties for their disclosure of coatings for inhalers and inhaler components. Other coatings, such as fluorinated ethylene propylene resins, or FEP, are also suitable. FEP is particularly suitable for use in coating canisters.

A first acceptable coating can be provided by the following method:

a) providing one or more component of the inhaler, such as the metered dose inhaler,

b) providing a primer composition comprising a silane having two or more reactive silane groups separated by an organic linker group,

c) providing a coating composition comprising an at least partially fluorinated compound,

d) applying the primer composition to at least a portion of the surface of the component,

e) applying the coating composition to the portion of the surface of the component after application of the primer composition.

The at least partially fluorinated compound will usually comprise one or more reactive functional groups, with the or each one reactive functional group usually being a reactive silane group, for example a hydrolysable silane group or a hydroxysilane group. Such reactive silane groups allow reaction of the partially fluorinated compound with one or more of the reactive silane groups of the primer. Often such reaction will be a condensation reaction.

One exemplary silane that can be used has the formula

X₃₋ₘ(R¹)ₘSi-Q-Si(R²)ₖX₃₋ₖ

wherein R¹ and R² are independently selected univalent groups, X is a hydrolysable or hydroxy group, m and k are independently 0, 1, or 2 and Q is a divalent organic linking group.

Useful examples of such silanes include one or a mixture of two or more of 1,2-bis(trialkoxysilyl) ethane, 1,6-bis(trialkoxysilyl) hexane, 1,8-bis(trialkoxysilyl) octane, 1,4-bis(trialkoxysilylethyl)benzene, bis(trialkoxysilyl)itaconate, and 4,4′-bis(trialkoxysilyl)-1,1′-diphenyl, wherein any trialkoxy group may be independently trimethoxy or triethoxy.

The coating solvent usually comprises an alcohol or a hydrofluoroether.

If the coating solvent is an alcohol, preferred alcohols are C₁ to C₄ alcohols, in particular, an alcohol selected from ethanol, n-propanol, or isopropanol or a mixture of two or more of these alcohols.

If the coating solvent is an hydrofluoroether, it is preferred if the coating solvent comprises a C₄ to C₁₀ hydrofluoroether. Generally, the hydrofluoroether will be of formula

C_{g}F_{2g+1}OCₕH₂ₕ₊₁

wherein g is 2, 3, 4, 5, or 6 and h is 1, 2, 3 or 4. Examples of suitable hydrofluoroethers include those selected from the group consisting of methyl heptafluoropropylether, ethyl heptafluoropropylether, methyl nonafluorobutylether, ethyl nonafluorobutylether and mixtures thereof.

The polyfluoropolyether silane can be of the formula

R^{f}Q¹ᵥ[Q²_{w}-[C(R⁴)₂-Si(X₃₋ₓ(R⁵)ₓ]_{y}]_{z}

wherein:

R^{f} is a polyfluoropolyether moiety;

Q¹ is a trivalent linking group;

each Q² is an independently selected organic divalent or trivalent linking group;

each R⁴ is independently hydrogen or a C₁₋₄ alkyl group;

each X is independently a hydrolysable or hydroxyl group;

R is a C₁₋₈ alkyl or phenyl group;

v and w are independently 0 or 1, x is 0 or 1 or 2; y is 1 or 2; and z is 2, 3, or 4.

The polyfluoropolyether moiety R^{f} can comprise perfluorinated repeating units selected from the group consisting of —(CₙF₂ₙO)—, —(CF(Z)O)—, —(CF(Z)CₙF₂ₙO)—, —(CₙF₂ₙCF(Z)O)—, —(CF₂CF(Z)O)—, and combinations thereof; wherein n is an integer from 1 to 6 and Z is a perfluoroalkyl group, an oxygen-containing perfluoroalkyl group, a perfluoroalkoxy group, or an oxygen-substituted perfluoroalkoxy group, each of which can be linear, branched, or cyclic, and have 1 to 5 carbon atoms and up to 4 oxygen atoms when oxygen-containing or oxygen-substituted and wherein for repeating units including Z the number of carbon atoms in sequence is at most 6. In particular, n can be an integer from 1 to 4, more particularly from 1 to 3. For repeating units including Z the number of carbon atoms in sequence may be at most four, more particularly at most 3. Usually, n is 1 or 2 and Z is an —CF₃ group, more wherein z is 2, and R^{f} is selected from the group consisting of —CF₂O(CF₂O)ₘ(C₂F₄O)ₚCF₂—, —CF(F₃)O(CF(CF₃)CF₂O)ₚCF(CF₃)—, —CF₂O(C₂F₄O)ₚCF₂—, —(CF)₂)₃(C₄F₈O)ₚ(CF₂)₃—, —CF(CF₃)—(OCF₂CF(CF₃))ₚO—CₜF₂ₜ—O(CF(CF₃)CF₂O)ₚCF(CF₃)—, wherein t is 2, 3 or 4 and wherein m is 1 to 50, and p is 3 to 40.

A cross-linking agent can be included. Exemplary cross-linking agents include tetramethoxysilane; tetraethoxysilane; tetrapropoxysilane; tetrabutoxysilane; methyl triethoxysilane; dimethyldiethoxysilane; octadecyltriethoxysilane; 3-glycidoxy-propyltrimethoxysilane; 3-glycidoxy-propyltriethoxysilane; 3-aminopropyl-trimethoxysilane; 3 -aminopropyl-triethoxysilane; bis(3 -trimethoxysilylpropyl) amine; 3-aminopropyl tri(methoxyethoxyethoxy) silane; N (2-aminoethyl)3-aminopropyltrimethoxysilane; bis (3-trimethoxysilylpropyl) ethylenediamine; 3-mercaptopropyltrimethoxysilane; 3-mercaptopropyltriethoxysilane; 3-trimethoxysilyl-propylmethacrylate; 3-triethoxysilypropylmethacrylate; bis(trimethoxysilyl) itaconate; allyltriethoxysilane; allyltrimethoxysilane; 3-(N-allylamino)propyltrimethoxysilane; vinyltrimethoxysilane; vinyltriethoxysilane; and mixtures thereof.

The component to be coated can be pre-treated before coating, such as by cleaning. Cleaning can be by way of a solvent, such as a hydrofluoroether, e.g. HFE72DE, or an azeotropic mixture of about 70% w/w trans-dichloroethylene; 30% w/w of a mixture of methyl and ethyl nonafluorobutyl and nonafluoroisobutyl ethers.

The above-described first acceptable coating is particularly useful for coating valves components, including one or more of valve stems, bottle emptiers, springs, and tanks. This coating system can be used with any type of inhaler and any formulation described herein. In embodiments the pharmaceutical performance of a MDI of the present invention is controlled so that it is similar to the pharmaceutical performance of a reference inhaler. For example, in embodiments the pharmaceutical performance of a MDI of the present invention is similar to that of Relvar® Ellipta® 100/25, a dry powder inhalation product that contains individual doses of 100 micrograms of fluticasone furoate and 25 micrograms of vilanterol. The dose of vilanterol in Relvar® Ellipta® 100/25 is given as a base equivalent, i.e., the dose is 25 micrograms of vilanterol base present in the form of vilanterol trifenatate. This is likewise true for the respective vilanterol doses of the other Relvar and Breo products described below. In embodiments the pharmaceutical performance of a MDI of the present disclosure is similar to that of Relvar® Ellipta® 200/25, a dry powder inhalation product that contains individual doses of 200 micrograms of fluticasone furoate and 25 micrograms of vilanterol. In embodiments the pharmaceutical performance of a MDI of the present disclosure is similar to that of Breo® Ellipta® 100/25, a dry powder inhalation product that contains individual doses of 100 micrograms of fluticasone furoate and 25 micrograms of vilanterol. In embodiments the pharmaceutical performance of a MDI of the present disclosure is similar to that of Breo® Ellipta® 200/25, a dry powder inhalation product that contains individual doses of 200 micrograms of fluticasone furoate and 25 micrograms of vilanterol. In embodiments the pharmaceutical performance of a MDI of the present disclosure is similar to that of Relvar® Ellipta® 92/22, a dry powder inhalation product that delivers a nominal dose of 92 micrograms of fluticasone furoate and 22 micrograms of vilanterol per inhalation. In embodiments the pharmaceutical performance of a MDI of the present disclosure is similar to that of Relvar® Ellipta® 184/22, a dry powder inhalation product that delivers a nominal dose of 184 micrograms of fluticasone furoate and 22 micrograms of vilanterol per inhalation. In embodiments the pharmaceutical performance of a MDI of the present disclosure is similar to that of Breo® Ellipta® 92/22, a dry powder inhalation product that delivers a nominal dose of 92 micrograms of fluticasone furoate and 22 micrograms of vilanterol per inhalation. In embodiments the pharmaceutical performance of a MDI of the present disclosure is similar to that of Breo® Ellipta® 184/22, a dry powder inhalation product that delivers a nominal dose of 184 micrograms of fluticasone furoate and 22 micrograms of vilanterol per inhalation.

Similar pharmaceutical performance may be evaluated by either in vitro or in vivo test methods.

Suitable in vitro test methods include, but are not limited to, single actuation content and aerodynamic particle size distribution. Single actuation content may be measured at beginning, middle, and/or end of life for an MDI using a flow rate of 28.3 L/min. U.S. Pharmacopeia (USP) <601> Apparatus A or other appropriate apparatus may be used. Aerodynamic particle size distribution may be measured at beginning, middle, and/or end of life using a flow rate of 28.3 L/min. The USP <601> Apparatus 1, Apparatus 6, or other appropriate apparatus may be used. Single actuation content may be further analyzed to determine fine particle mass (FPM) and/or impactor stage mass (ISM). Aerodynamic particle size distribution may be further analyzed to determine mass median aerodynamic diameter (MMAD).

Suitable in vivo test methods include, but are not limited to, pharmacokinetic (PK) bioequivalence studies and clinical pharmacodynamic bioequivalence studies. One of ordinary skill in the art will understand the appropriate parameters and ranges of performance that are required to establish bioequivalence. An exemplary PK bioequivalence study will be considered to have established bioequivalence if the area under the curve (AUC) and the Cₘₐₓ (maximum concentration) of active and/or active metabolite in the plasma has a 90% confidence interval for the geometric mean of the ratio between test and reference articles that falls within limits of 80 to 125%. An exemplary clinical pharmacodynamic bioequivalence study will establish bioequivalence if one or more clinical measures of lung function, such as FEV1, have a 90% confidence interval for the mean of the ratio between test and reference articles that falls within limits of 80 to 125%.

### LIST OF EXEMPLARY EMBODIMENTS

The following embodiments are meant to be illustrative, and are not intended to be limiting unless otherwise specified.

1. A composition comprising

particulate fluticasone or a pharmaceutically acceptable salt or solvate thereof

particulate vilanterol trifenatate; and

1,1-difluoroethane (HFA-152a).

2. The composition of embodiment 1, wherein the fluticasone or a pharmaceutically acceptable salt or solvate thereof is fluticasone furoate.

3. The composition of any previous embodiment, wherein the vilanterol or a pharmaceutically acceptable salt or solvate thereof is vilanterol trifenatate.

4. The composition of any previous embodiment, wherein the propellant consists essentially of 1,1-difluoroethane (HFA-152a).

5. The composition of any previous embodiment, wherein the canister size of the fluticasone is between about 2 micrometers and 4 micrometers.

6. The composition of any previous embodiment, wherein the canister size of the vilanterol trifenatate is between about 1 micrometer and 2 micrometers.

7. The composition of any previous embodiment, wherein the concentration of the fluticasone is between about 1.0 mg/g and 2.5 mg/g.

8. The composition of any previous embodiment, wherein the concentration of the fluticasone is between about 2.0 mg/g and 4.5 mg/g.

9. The composition of any previous embodiment, wherein the concentration of the vilanterol trifenatate is between about 0.2 mg/g and 1.0 mg/g.

10. A composition comprising

particulate fluticasone or a pharmaceutically acceptable salt or solvate thereof;

particulate vilanterol or a pharmaceutically acceptable salt or solvate thereof; and

1,1-difluoroethane (HFA-152a), wherein fluticasone and vilanterol or the pharmaceutically acceptable salts or solvates thereof are the only active medicaments in the composition.

11. The composition of embodiment 10, wherein the fluticasone or a pharmaceutically acceptable salt or solvate thereof is fluticasone furoate. 12. The composition of any of embodiments 10-11, wherein the vilanterol or a pharmaceutically acceptable salt or solvate thereof is vilanterol trifenatate.

13. The composition of any of embodiments 10-12, wherein the propellant consists essentially of 1,1-difluoroethane (HFA-152a).

14. The composition of any of embodiments 10-13, wherein the canister size of the fluticasone is between about 2 micrometers and 4 micrometers.

15. The composition of any of embodiments 10-14, wherein the canister size of the vilanterol trifenatate is between about 1 micrometer and 2 micrometers.

16. The composition of any of embodiments 10-15, wherein the concentration of the fluticasone is between about 1.0 mg/g and 2.5 mg/g.

17. The composition of any of embodiments 10-16, wherein the concentration of the fluticasone is between about 2.0 mg/g and 4.5 mg/g.

18. The composition of any of embodiments 10-17, wherein the concentration of the vilanterol trifenatate is between about 0.2 mg/g and 1.0 mg/g.

19. An aerosol canister comprising a composition of any preceding embodiment.

20. The aerosol canister of embodiment 19 comprising at least one surface having a primer composition comprising a silane having two or more reactive silane groups separated by an organic linker group disposed thereon, wherein the primer composition has a coating composition comprising an at least partially fluorinated compound disposed thereon.

21. The aerosol canister of embodiment 20, wherein the at least partially fluorinated compound is a polyfluoropolyether silane.

22. The aerosol canister of embodiments 20 or 21, wherein the at least one surface is at least a portion of a valve surface.

23. An inhaler comprising the composition of any one of embodiments 1 to 18 or the aerosol canister of any one of embodiments 19 to 23.

### EXAMPLES

1,1-difluoroethane (HFA-152a) was obtained from Mexichem (Runcorn, UK). Vilanterol trifenatate was obtained from Hovione (Portugal). Fluticasone furoate was obtained from Hovione (Portugal).

### Example 1

Metered dose inhalers (MDIs) were prepared using 16 mL aluminum canisters coated with FEP (IntraPac International, Mooresville, N.C., USA), 63 microliter 3M retention type valves with a PBT (polybutylene terephthalate) stem and EPDM (ethylene-propylene diene terpolymer elastomer) diaphragm seals (3M Corporation), and 0.25 mm exit orifice diameter 3M Mk6 actuators (Oechsler, Ansbach, Germany) fitted with an integrated dose counter. The valves were coated with a fluoropolymer coating according to the general process described in Example 2 of U.S. Patent Application Publication 2017/0152396 A1, Jinks et al. Vilanterol trifenatate was micronized to provide a mass median diameter (MMD) of about 1.5 microns. Fluticasone furoate was high pressure homogenized to provide a mass median diameter (MMD) range of about 3.5 microns. The canisters were cold filled with a suspension formulation having 0.1744% fluticasone furoate, 0.0698% vilanterol trifenatate, and 99.7558% HFA-152a. The bulk formulation for cold filling individual canisters was prepared by combining fluticasone furoate and vilanterol trifenatate with the HFA-152a propellant in a vessel chilled to less than −40° C. The suspension was sonicated for 10 minutes to disperse the suspension.

### Delivered Dose Studies

The start of unit life delivered dose was determined using standard unit spray collection apparatus (USCA) fitted with a filter. For each determination, an MDI was attached to the USCA using a coupler and actuated a single time. Immediately prior to attachment, the MDI was vigorously shaken. Prior to collection of the test sample, the MDI was primed by actuating four times. Prior to each priming shot the MDI was vigorously shaken. The flow rate through the equipment was regulated to 28.3 L/min +/−0.5 L/min. The test sample deposited in the USCA was collected by rinsing with a known volume of collection solvent. The recovered samples were then analyzed for sample content using an HPLC assay with reference to a known standard. An HPLC instrument with a UV detector (220 nm at 0 minutes, 240 nm at 5 minutes) and a symmetry shield RP18, 150 mm×4.6 mm (3.5 μm) column (temperature 25° C.) was used. The mobile phase was 10 mM SDS (sodium dodecyl sulphate), 60:40 (v/v) acetetonitrile:50 mM NH4OAc (ammonium acetate), pH 5.50. The injection volume was 50 μl and the flow rate was 1.0 mL/min.

Start of life dose content uniformity for fluticasone furoate was 90.9 mcg/actuation and for vilanterol was 23.1 mcg/actuation.

### Next Generation Impactor (NGI) Studies

The aerodynamic particle size distribution emitted from each MDI was evaluated using a Next Generation Impactor Instrument (MSP Corporation, Shoreview, Minn.). For each test, an MDI was attached to the throat component (Emmace anatomical throat, Emmace Consulting, Lund, Sweden) of the NGI instrument and actuated 6 times into the instrument. Prior to each actuation the MDI was vigorously shaken Immediately prior to attachment, the MDI was primed by actuating 4 times. Prior to each priming shot the MDI was vigorously shaken. The flow rate through the instrument during testing was regulated at 30 L/minute. The test sample (fluticasone furoate and vilanterol trifenatate) deposited on the valve stem, actuator, throat assembly (Emmace anatomical throat), individual uncoated collection cups 1-7, micro-orifice collector (MOC), and final filter component was collected by rinsing each individual component with a known volume of collection solvent. The recovered samples were then analyzed for sample content using an HPLC assay with reference to a known standard. An HPLC instrument with a UV detector (220 nm at 0 minutes, 240 nm at 5 minutes) and a symmetry shield RP-18, 4.6-150 mm column (25 ° C. column temp) was used. The mobile phase was 10 mM SDS, 60:40 (v/v) acetonitrile:50 mM NH₄OAc, pH 5.5). The injection volume was 50 microliters and the flow rate was 1.0 mL/min.

In Table 1 the fine particle mass (FPM), impactor sized mass (ISM), mass median aerodynamic diameter (MMAD), and throat hold up data for fluticasone furoate (FF) and vilanterol (V) is presented. Three individual MDIs were tested and the result is presented as the mean value.

Throat hold up was determined as the ratio of the sample content from the throat assembly divided by the total ex-actuator content.

TABLE 1

| API | FPM (mcg) | ISM (mcg) | MMAD (um) | Throat hold up (%) |
|---|---|---|---|---|
| FF | 21.6 | 29.7 | 3.3 | 70.8 |
| V | 9.2 | 10.1 | 1.9 | 61.4 |

Fine particle mass (FPM) was calculated using CITDAS (Copley Inhaler Testing Data Analysis Software, from Copley Scientific, Nottingham, UK) as the sum of sample content with a particle size less than 5 micrometers (um), reported as micrograms per actuation (mcg/act).

Median mass aerodynamic diameter (MMAD) was calculated using CITDAS (Copley Inhaler Testing Data Analysis Software).

Impactor sized mass (ISM) was determined as the sum of the sample content determined for Cups 2-7, the MOC and the filter, reported as micrograms per actuation (mcg/act).

### Example 2

Metered dose inhalers (MDIs) were prepared using 16 mL aluminum canisters coated with FEP (IntraPac International, Mooresville, N.C., USA), 25 microliter 3M retention type valves with a PBT stem and EPDM diaphragm seals (3M Corporation), and 3M Mk6 actuators with a 0.25 mm exit orifice diameter, 0.8 mm jet length. The actuators contained an integrated dose counter. The valves were coated with a fluoropolymer coating according to the general process described in Example 2 of U.S. Patent Application Publication 2017/0152396 A1, Jinks et al. Vilanterol trifenatate was micronized to provide a mass median diameter (MMD) of about 1.5 microns. Fluticasone furoate was high pressure homogenized to provide a mass median diameter (MMD) range of about 3.5 microns. The canisters were cold filled with a suspension formulation having 0.8791 wt. % fluticasone furoate, 0.1758 wt. % vilanterol trifenatate, and 98.9451 wt. % HFA-152a. In the filling procedure fluticasone furoate and vilanterol trifenatate were added to each canister followed by the addition of chilled (about −55° C. to −60° C.) HFA-152a. Each canister with valve was crimped. and then sonicated for 10 minutes to disperse the suspension. The assembled MDIs were tested according to the NGI Studies procedure described above. For fluticasone furoate, the calculated FPM was 104.9 micrograms/actuation and the calculated MMAD was 3.5 micrometers. For vilanterol, the calculated FPM was 13.6 micrograms/actuation and the calculated MMAD was 2.6 micrometers.

### Example 3

MDIs were prepared according to the procedure described in Example 2 with the exception that a 50 microliter valve was used and the formulation contained 0.4396 wt. %

fluticasone furoate, 0.0879 wt. % vilanterol trifenatate, and 99.4725 wt. % HFA-152a. The assembled MDIs were tested according to the NGI Studies procedure described above. For fluticasone furoate, the calculated FPM was 69.3 micrograms/actuation and the calculated MMAD was 3.4 micrometers. For vilanterol, the calculated FPM was 8.8 micrograms/actuation and the calculated MMAD was 2.7 micrometers.

### Example 4

MDIs were prepared according to the procedure described in Example 2 with the exception that a 63 microliter valve was used and the formulation contained 0.3489 wt. % fluticasone furoate, 0.0698 wt. % vilanterol trifenatate, and 99.5813 wt. % HFA-152a. The MDIs were tested according to the NGI Studies procedure described above. For fluticasone furoate, the calculated FPM was 55.9 micrograms/actuation and the calculated MMAD was 3.3 micrometers. For vilanterol, the calculated FPM was 6.7 micrograms/actuation and the calculated MMAD was 2.3 micrometers.

### Example 5

MDIs were prepared according to the procedure described in Example 2 with the exception that an uncoated 100 microliter valve was used and the formulation contained 0.2198 wt. % fluticasone furoate, 0.0440 wt. % vilanterol trifenatate, and 99.7362 wt. % HFA-152a. The assembled MDIs were tested according to the NGI Studies procedure described above. For fluticasone furoate, the calculated FPM was 45.2 micrograms/actuation and the calculated MMAD was 3.3 micrometers. For vilanterol, the calculated FPM was 5.5 micrograms/actuation and the calculated MMAD was 2.5 micrometers.

### Example 6

MDIs were prepared according to the procedure described in Example 2 with the exception that the formulation contained 0.4396 wt. % fluticasone furoate, 0.1758 wt. % vilanterol trifenatate, and 99.3846 wt. % HFA-152a. The assembled MDIs were tested according to the NGI Studies procedure described above. For fluticasone furoate, the calculated FPM was 41.0 micrograms/actuation and the calculated MMAD was 3.1 micrometers. For vilanterol, the calculated FPM was 11.4 micrograms/actuation and the calculated MMAD was 2.1 micrometers.

### Example 7

MDIs were prepared according to the procedure described in Example 2 with the exception that a 50 microliter valve was used and the formulation contained 0.2198 wt. %

fluticasone furoate, 0.0879 wt. % vilanterol trifenatate, and 99.6923 wt. % HFA-152a. The assembled MDIs were tested according to the NGI Studies procedure described above. For fluticasone furoate, the calculated FPM was 26.3 micrograms/actuation and the calculated MMAD was 2.9 micrometers. For vilanterol, the calculated FPM was 7.7 micrograms/actuation and the calculated MMAD was 2.2 micrometers.

### Example 8

MDIs were prepared according to the procedure described in Example 2 with the exception that a 63 microliter valve was used and the formulation contained 0.1744 wt. % fluticasone furoate, 0.0698 wt. % vilanterol trifenatate, and 99.7558 wt. % HFA-152a. The assembled MDIs were tested according to the NGI Studies procedure described above. For fluticasone furoate, the calculated FPM was 23.8 micrograms/actuation and the calculated MMAD was 3.0 micrometers. For vilanterol, the calculated FPM was 5.9 micrograms/actuation and the calculated MMAD was 2.3 micrometers.

### Example 9

MDIs were prepared according to the procedure described in Example 2 with the exception that an uncoated 100 microliter valve was used and the formulation contained 0.1099 wt. % fluticasone furoate, 0.0440 wt. % vilanterol trifenatate, and 99.8461 wt. % HFA-152a. The assembled MDIs were tested according to the NGI Studies procedure described above. For fluticasone furoate, the calculated FPM was 19.3 micrograms/actuation and the calculated MMAD was 3.2 micrometers. For vilanterol, the calculated FPM was 4.2 micrograms/actuation and the calculated MMAD was 2.6 micrometers.

All references and publications cited herein are expressly incorporated herein by reference in their entirety into this disclosure.

Various features and aspects of the present disclosure are set forth in the following claims.

1. A composition comprising:
particulate fluticasone or a pharmaceutically acceptable salt or solvate thereof;
particulate vilanterol or a pharmaceutically acceptable salt or solvate thereof; and
1,1-difluoroethane (HFA-152a).

2. The composition of claim 1, wherein the fluticasone or a pharmaceutically acceptable salt or solvate thereof is fluticasone furoate.

3. The composition of claim 1, wherein the vilanterol or a pharmaceutically acceptable salt or solvate thereof is vilanterol trifenatate.

4. The composition of claim 1, wherein the propellant consists essentially of 1,1-difluoroethane (HFA-152a).

5. The composition of claim 1 contained in a canister, wherein the size of the fluticasone particles in the canister is between about 2 micrometers and 4 micrometers.

6. The composition of claim 1 contained in a canister, wherein the size of the vilanterol trifenatate particles in the canister is between about 1 micrometer and 2 micrometers.

7-9. (canceled)

10. A composition comprising
particulate fluticasone or a pharmaceutically acceptable salt or solvate thereof;
particulate vilanterol or a pharmaceutically acceptable salt or solvate thereof; and
1,1-difluoroethane (HFA-152a), wherein fluticasone and vilanterol or the pharmaceutically acceptable salts or solvates thereof are the only active medicaments in the composition.

11. The composition of claim 10, wherein the fluticasone or a pharmaceutically acceptable salt or solvate thereof is fluticasone furoate.

12. (canceled)

13. The composition of claim 10, wherein the propellant consists essentially of 1,1-difluoroethane (HFA-152a).

14. The composition of claim 10 contained in a canister, wherein the size of the fluticasone particles in the canister is between about 2 micrometers and 4 micrometers.

15. The composition of claim 10 contained in a canister, wherein the size of the vilanterol trifenatate particles in the canister is between about 1 micrometer and 2 micrometers.

16-18. (canceled)

19. An aerosol canister comprising the composition of claim 1.

20. The aerosol canister of claim 19 comprising at least one surface having a primer composition comprising a silane having two or more reactive silane groups separated by an organic linker group disposed thereon, wherein the primer composition has a coating composition comprising an at least partially fluorinated compound disposed thereon.

21. (canceled)

22. The aerosol canister of claim 20, wherein the at least one surface is at least a portion of a valve surface.

23. An inhaler comprising the composition of claim 1.

24. An aerosol canister comprising the composition of claim 10.

25. The aerosol canister of claim 25 comprising at least one surface having a primer composition comprising a silane having two or more reactive silane groups separated by an organic linker group disposed thereon, wherein the primer composition has a coating composition comprising an at least partially fluorinated compound disposed thereon.

26. The aerosol canister of claim 25, wherein the at least partially fluorinated compound is a polyfluoropolyether silane.

27. An inhaler comprising the composition of claim 10.

28. An inhaler comprising the aerosol canister of claim 19.
